(19) United States
(12) Patent Application Publication
(10) Pub. No.: US 2014/0288102 A1
(43) Pub. Date: Sep. 25, 2014

(54) PREVENTION OF RECURRENCES OF URETHRAL STRICTURES FOLLOWING CONVENTIONAL THERAPY

(71) Applicant: Mehdi JAIDANE, Sousse (TN)

(72) Inventor: Mehdi JAIDANE, Sousse (TN)

(73) Assignee: Alain LEBET, Lausanne (CH)

(21) Appl. No.: 14/222,941

(22) Filed: Mar. 24, 2014

Related U.S. Application Data
(63) Continuation of application No. 12/739,324, filed on Apr. 22, 2010, now abandoned.

(30) Foreign Application Priority Data
Oct. 23, 2007 (FR) 07/07387

(51) Int. Cl. A61K 9/00; A61K 45/06; A61K 31/517

(52) U.S. Cl. CPC A61K 9/0034; A61K 31/517; A61K 45/06
USPC 514/266.22

(57) ABSTRACT

The instant invention relates to the prevention of recurrence of urethral stricture after a conventional treatment. Compositions applicable to the prevention of recurrence of urethral stricture after as conventional treatment, including a pharmaceutically effective amount of halofuginone, are disclosed. Urethral stricture, as common disease, appears secondary to urethritis, urethral infection, urethral inflammation, urethral instrumentation, urethral catheterization, urethral trauma, urethral surgery and all types of urethral lesions. Conventional treatments by internal urethrotomy, urethral dilatation or surgical urethroplasty are available and can cure urethral stricture, but with a relatively high rate of recurrence of the stricture. Halofuginone can prevent the recurrence of urethral stricture after conventional treatment via internal urethrotomy, urethral dilatation or surgical urethroplasty.

This application is a Continuation of U.S. application Ser. No. 12/739,324, which is the National Stage of International Application PCT/IB2008/003375, filed Oct. 23, 2008, which claims priority to French Application No. 07/07387, filed Oct. 23, 2007. The disclosures of application Ser. No. 12/739,324 and PCT/IB2008/003375 are expressly incorporated herein by reference in their entireties.

### FIELD AND BACKGROUND OF THE INVENTION

The present invention relates to a composition useful for preventing recurrences of urethral strictures after any other type of conventional therapy such as endoscopic internal urethrotomy, urethral dilatation, or surgical urethroplasty.

Urethral stricture is a common clinical condition characterized by stenosis of the urethral lumen due to the growth of sclerous tissue, generally as a consequence of the scarring of an urethral injury. The most common causes of urethral stricture are instrumentation and catheterization of the urethra, external trauma and urethral infection. The most widely-used therapies for urethral stricture disease, i.e. endoscopic internal urethrotomy and urethral dilatation, are effective in treating urethral strictures in the short run. These therapies allow first-intent treatment to be given rapidly to large numbers of patients, albeit with high recurrence rates (Stormont T J et al., Journal of Urology, volume 150 (5 Pt 2), pp. 1725-8, November 1993; Holm-Nielsen A et al., British Journal of Urology, volume 56, p. 308, 1984). Moreover, urethral stricture recurrence rates increase with the duration of progression and the repetition of procedures (Heyns C F et al., Journal of Urology, volume 160 (2), p. 356-358, August 1998).

Surgical urethroplasty techniques are also associated with high cure rates, reaching 85-90% in some series (Barbagli G et al., Journal of Urology, volume 155, pp. 1918-1919, June 2011; Webster G D et al., Journal of Urology, volume 134, p. 892, 1985), but these procedures are often complicated and require specific training.

Significant rates of urethral stricture recurrence are also observed after these primary surgical procedures.

Several techniques have been used in order to decrease recurrence rates following internal urethrotomy: prolonged urethral catheterization, placement of urethral stents (Jordan G H et al., pp. 3346-3347, in Walsh et al.: Campbell's Urology, 7th Edition, Philadelphia, W B Saunders, 1898; Milroy E, Journal of Urology, volume 150, pp. 1729-1733, 1993) or intermittent self-catheterization (Harriss D R et al., British Journal of Urology, volume 74, p. 790, 1994; Kjaergaard B et al., British Journal of Urology, volume 73, p. 692, 1994; Bodker A et al., Journal of Urology, volume 148, p. 308, 1992), with various results. All these techniques are invasive and not commonly used.

As compared to the normal urethra, the quantification of the different subtypes of collagen in the fibrous tissue of the urethral stricture evidences changes in the ratio of collagen subtypes and in the relative proportion of type I and type III collagen. With regard to the proportion of type I collagen and type III collagen, the proportion of type I collagen is increased in the tissue of the urethral stricture, with a correlated decrease in the proportion of type III collagen. Both subtypes of collagen differ by their mechanical properties, and changes in the collagen III:I ratio have been shown to alter the tissue compliance (Baskin L S et al., British Journal of Urology, volume 150, p. 642, 1993).

Drugs modulating the synthesis of a given subtype of collagen may thus be useful for preventing onset or recurrence of urethral strictures. In particular, a subtype-specific inhibitor of collagen synthesis could be useful for inhibiting the process leading to urethral stenosis.

This specific inhibitor is a composition comprising a pharmaceutically effective amount of a pharmaceutically active compound of formula:

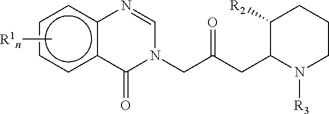

wherein

R1 is selected from the group consisting of hydrogen, halogen, nitro, benzo, lower alkyl, phenyl and lower alkoxy;

R2 is selected from the group consisting of hydroxy, acetoxy and lower alkoxy,

R3 is selected from the group consisting of hydrogen and lower alkenoxy-carbonyl; and

n is either 1 or 2;

and pharmaceutically acceptable salts thereof.

Within this group of compounds, Halofuginone has been found to be particularly effective for this kind of treatment.

In addition, the inventor has demonstrated for the first time the effectiveness of Halofuginone in preventing the recurrence of urethral strictures after endoscopic internal urethrotomy (Jaidane et al., Journal of Urology, volume 170, pp. 2049-2052, November 2003). This preventive effect on the recurrence of urethral stricture after successful treatment by endoscopic internal urethrotomy had never been demonstrated before this work.

Thus, Halofuginone can prevent the recurrence of urethral strictures after endoscopic internal urethrotomy or a similar treatment such as urethral dilatation.

There is a recognized medical need for an inhibitor of urethral stricture recurrences following conventional therapy.

### SUMMARY OF THE INVENTION

According to the present invention, there is provided a composition allowing the recurrence of urethral stricture following conventional first-line therapy such as endoscopic internal urethrotomy, urethral dilatation and surgical urethroplasty to be prevented. The composition comprises a pharmaceutically effective amount of a compound in combination with a pharmaceutically acceptable carrier, said compound having the formula:

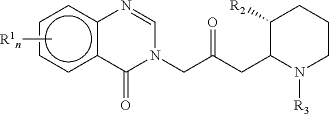

wherein

R1 is selected from the group consisting of hydrogen, halogen, nitro, benzo, lower alkyl, phenyl and lower alkoxy;

R2 is selected from the group consisting of hydroxy, acetoxy and lower alkoxy,

R3 is selected from the group consisting of hydrogen and lower alkenoxy-carbonyl; and

n is either 1 or 2;

and pharmaceutically acceptable salts thereof.

Within this group of compounds, the preferred compound is Halofuginone.

According to another embodiment of the present invention, there is provided a composition for preventing the recurrence of urethral strictures following conventional first-line therapy such as internal urethrotomy, urethral dilatation and surgical urethroplasty, comprising administering to a subject a pharmaceutically effective amount of a compound having the formula:

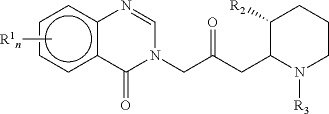

wherein

R1 is selected from the group consisting of hydrogen, halogen, nitro, benzo, lower alkyl, phenyl and lower alkoxy;

R2 is selected from the group consisting of hydroxy, acetoxy and lower alkoxy,

R3 is selected from the group consisting of hydrogen and lower alkenoxy-carbonyl; and

n is either 1 or 2;

and pharmaceutically acceptable salts thereof.

In all these embodiments, the preferred compound is Halofuginone. Hereinafter, the term “Halofuginone” is defined as a compound having the formula:

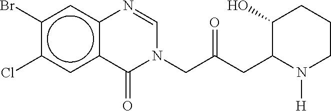

and pharmaceutically acceptable salts thereof. The composition preferably includes a pharmaceutically acceptable carrier for the compound.

Preferably, all of the compounds referred to hereinabove can be either the compound per se, as described by the formula, and/or pharmaceutically acceptable salts thereof.

### BRIEF DESCRIPTION OF THE FIGURES

FIG. 1A shows a Hematoxylin- and Eosin-stained urethra at the site of a stricture showing severe fibrosis in a control rabbit receiving a halofuginone-free diet.

FIG. 1B shows Type I collagen fibers in a Sirius Red-stained urethra at the site of a stricture showing severe fibrosis in a control rabbit receiving a halofuginone-free diet.

FIG. 2A shows a retrograde urethrocystogram taken in a rabbit of the study group receiving Halofuginone prior to internal urethrotomy.

FIG. 2B shows a retrograde urethrocystogram taken in a rabbit of the study group receiving Halofuginone at 10 weeks after internal urethrotomy.

FIG. 3A shows a Hematoxylin- and Eosin-stained urethra at the site of prior internal urethrotomy in a rabbit of the study group receiving Halofiginone.

FIG. 3B shows Type I collagen fibers in a Sirius Red-stained urethra at the site of prior internal urethrotomy in a rabbit of the study group receiving Halofiginone.

### DESCRIPTION OF THE PREFERRED EMBODIMENTS

Unexpectedly, Halofuginone has been found to effectively inhibit the recurrence of urethral strictures following internal urethrotomy. Such an effect had not been foreseen by the prior art. In fact, no other substance had been previously described as preventing recurrence of urethral strictures following internal urethrotomy or urethral dilatation. Furthermore, the teachings of the prior art did not include the prevention of the recurrence of urethral strictures by Halofuginone.

Consequently, as described in detail below, Halofuginone may be used as a treatment for preventing recurrence of urethral strictures following primary therapy by internal urethrotomy, urethral dilatation or surgical urethroplasty.

### EXAMPLE 1

Halofuginone for Preventing the Renewed Onset of Urethral Strictures:

Near-circumferential, 15 mm long electrocoagulation of the bulbar urethra was performed endoscopically on 20 New Zealand male rabbits using a pediatric resectoscope and a round-ended electrocautery. The rabbits were randomized into 2 groups of 10 animals each. The first group received a diet containing 10 mg/kg of Halofuginone initiated 4 days before electrocoagulation and continued for 3 weeks.

The second group received a standard chow, free of Halofuginone.

Three weeks after electrocoagulation, the rabbits were examined by video urethrocystoscopy and retrograde urethrocystography and subsequently sacrificed for histological examination. The urethra was removed in one piece and fixed in 10% buffered formaldehyde. The histological examination was performed after the specimens were embedded in paraffin and stained with Masson trichrome, hematoxylin-eosin, and Sirius Red (IMEB, Inc., Chicago, Ill.) in order to evaluate fibrosis. With the latter staining method, collagen appears in red.

All rabbits in the control group had significant stricture of the urethra. The urethral lumen was reduced by 70-95% (mean 87.5%). The strictures were 9 to 18 mm in length (mean 12 mm). In these rabbits, the histological studies revealed a regenerating urothelium covering the walls of the urethral lumen and thick hyaline fibrosis located in the submucosal and smooth muscle layers, sometimes extending to the adventitia. Fibrosis was severe in all rabbits.

In contrast, only 2 out of the 10 Halofuginone-treated rabbits had significant urethral stricture. In these strictures, the reduction in urethral lumen caliber was 70% and 90%, respectively. These strictures were respectively 10 and 8 mm in length. Two other rabbits showed narrowing of the urethral lumen less than 30%. At the site of the stricture, the urethra was rigid and white to yellow in colour. The urethra was normal in all 6 remaining rabbits. In both rabbits with significant urethral strictures, the histological pattern was similar to that found in the rabbits of the control group. In the other rabbits of the Halofuginone treatment group, the histological study showed only focal alterations, with limited areas of fibrosis alternating with subnormal or normal urethral walls. The difference between both groups regarding the number of rabbits with stricture was statistically significant (p<0.001).

Table 1 shows this preventive effect of Halofuginone on the onset of urethral stricture after this experimental urethral injury.

TABLE 1
Preventive effect of Halofuginone on the onset of
urethral strictures after urethral injury.

| Variable | Halofuginone | Control group | P Value |
|---|---|---|---|
| Nr. of rabbits | 10 | 10 | |
| Nr. of urethral | 2 | 10 | <0.001 |
| strictures | | | |

### EXAMPLE 2

Halofuginone for Preventing the Recurrence of Urethral Strictures Following Internal Urethrotomy:

Near-circumferential, 15 mm long electrocoagulation of the bulbar urethra was performed endoscopically on 48 New Zealand male rabbits using a pediatric resectoscope and a round-ended electrocautery. Three weeks after electrocoagulation, the urethral strictures were evaluated using video urethrocystoscopy and retrograde urethrocystography. All surviving rabbits showed significant urethral strictures. The urethral lumen was reduced by 70-95% (mean 88.5%). The obtained strictures were 7 to 19 mm in length (mean 11.7). Endoscopic internal urethrotomy was performed on all surviving rabbits using a cold knife, under visual guidance with a 0.028-inch diameter endoscopic guidewire. A single deep incision was made at the 12 o'clock position in all animals.

The rabbits were subsequently randomized into 2 groups: one study group receiving Halofuginone for 10 weeks starting on the day of the urethrotomy, and one control group without Halofuginone. The study group was fed on a diet containing 10 mg/kg of Halofuginone, whereas the control group received a normal, Halofuginone-free diet.

The rabbits were then monitored and evaluated for urethral strictures if any of the following signs appeared: decrease in 24-hour urine output, anorexia, worsening of general health status with palpable bladder distension. In the absence of any of these signs, the animals were systematically evaluated for urethral strictures 10 weeks after urethrotomy. This evaluation was performed using video urethrocystoscopy and retrograde urethrocystography. Thereafter, the rabbits were sacrificed for histological examination.

The entire urethra was removed and fixed in 10% buffered formaldehyde. The histological examination was performed after the specimens had been embedded in paraffin and stained with Masson trichrome, hematoxylin-eosin, and Sirius Red (IMEB, Inc., Chicago, Ill.) in order to evaluate fibrosis. With the latter staining method, collagen appears in red.

The results are shown in Table 2. During the first week after electrocoagulation, 3 rabbits out of 48 died. The number of rabbits in the study and control groups was 22 and 23, respectively. Regarding the strictures obtained following electrocoagulation, both groups were comparable in terms of lumen caliber reduction and length of strictures. Two rabbits died immediately after endoscopic internal urethrotomy due to urethral perforation. Another 3 rabbits per group died during follow-up from causes unrelated to the experimental setting. Thus, at the end of the experiment, 18 and 19 evaluable rabbits remained in the study group and control group, respectively.

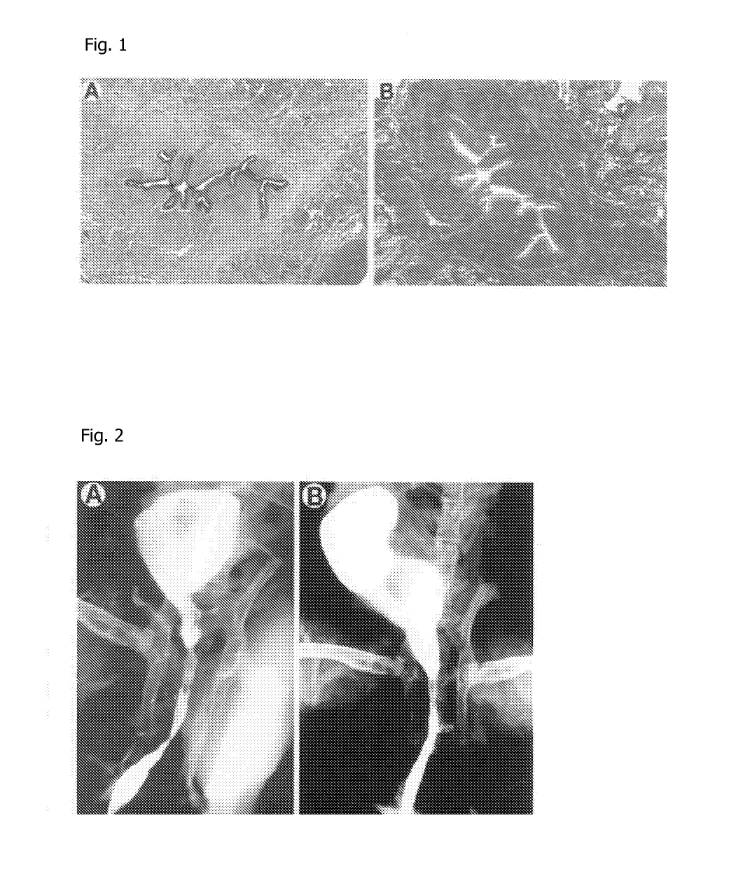

In the control group, the urethral stricture recurrence rate was higher than in the study group, and this difference was statistically significant (see Table 2). In the control group, recurrence of urethral stricture was suspected based on clinical signs, and this was confirmed by retrograde urethrocystography and video urethrocystoscopy in 6 rabbits, on average 19.6 days after urethrotomy (range 11-36 days). In all other rabbits, stricture was diagnosed at 10 weeks. FIG. 1 shows the histological appearance of a severe stricture of the urethra in 1 control rabbit.

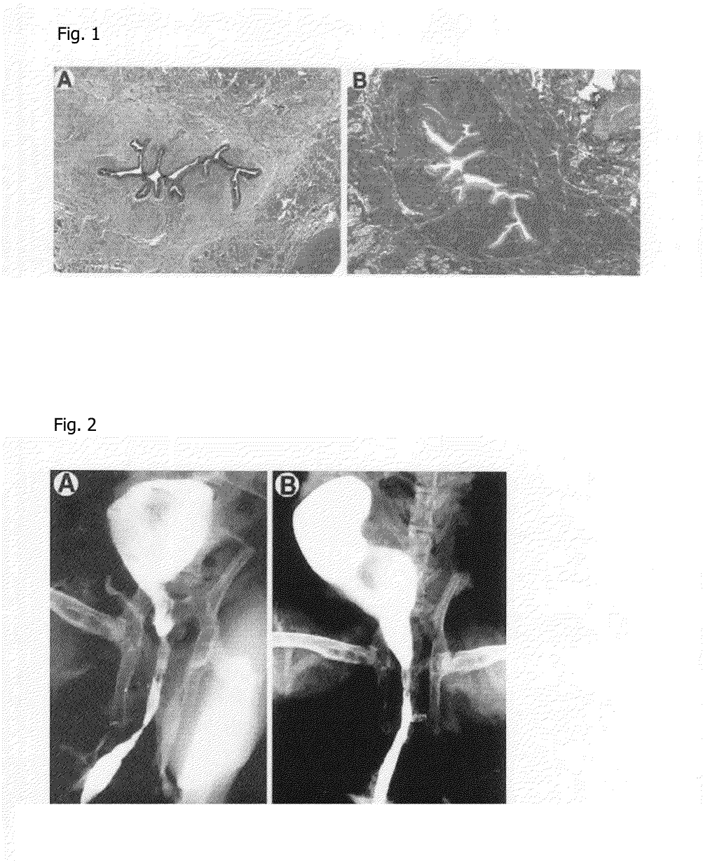

In the study group, 13 rabbits developed no recurrence of urethral stricture (FIG. 2). Based on clinical signs, recurrence of urethral stricture was suspected in 3 other rabbits, and this was confirmed radiologically and endoscopically 12 to 43 days after urethrotomy. In the remaining 2 rabbits, recurrent stricture was diagnosed at the 10-week evaluation.

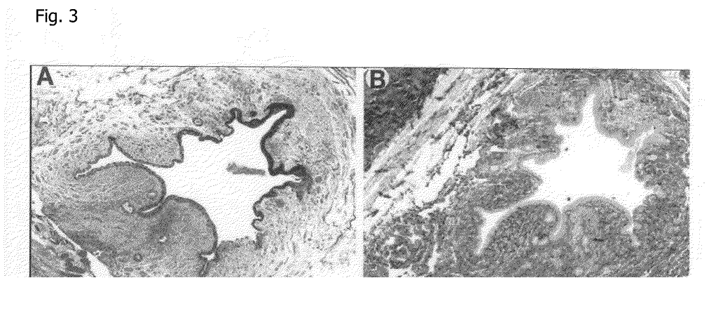

In the rabbits with no recurrence of stricture, the histological study of the site of the prior urethrotomy revealed no fibrosis or minimal fibrosis (FIG. 3).

TABLE 2
Effect of Halofuginone on the recurrence of urethral
stricture after endoscopic interal urethrotomy

| | Group receiving | | |
|---|---|---|---|
| Variable | Halofuginone | Control group | P value |
| Nr. of rabbits | 18 | 19 | |
| Appearance of post- | | | |
| coagulation stricture | | | |
| Average length (mm) | 11.4 | 11.6 | 0.85 |
| Average reduction of lumen | 88.3 | 88.4 | 0.89 |
| (%) | | | |
| Post-urethrotomy recurrent | | | |
| stricture | | | |
| Nr./total Nr. (%) | 5/18 (27) | 14/19 (73) | 0.005 |
| Average length (mm) | 7.2 | 11.4 | |
| Average reduction of lumen | 92 | 82.9 | |
| (%) | | | |
| Nr. according to extent of | | | |
| histological fibrosis | | | |
| Severe | 4 | 12 | |
| Moderate to severe | 1 | 2 | |
| Mild or absent | 13 | 5 | |

### DETAILED DESCRIPTION OF APPENDED DRAWINGS

FIG. 1 shows a cross-sectional microphotograph of the urethra at the site of the stricture showing severe fibrosis in a control rabbit receiving a halofuginone-free diet. (A) Hematoxylin and Eosin. (B) Type I collagen fibers appearing in red following staining with Sirius Red.

FIG. 2 shows shows a retrograde urethrocystogram taken in a rabbit of the study group receiving Halofuginone. (A) Prior to internal urethrotomy, significant stricture of the bulbar urethra was observed. (B) At 10 weeks, the bulbar urethra appeared normal in caliber and no recurrent stricture was seen.

FIG. 3 shows a microphotograph of a cross-section of the urethra at the site of prior internal urethrotomy showing minimal fibrosis in a rabbit of the study group receiving Halofuginone. (A) Hematoxylin and Eosin. (B) Type I collagen fibers appearing in red following staining with Sirius Red.

### EXAMPLE 3

Suitable Formulations for the Administration of Halofuginone:

Halofuginone can be given to a subject by various routes, all well-known in the art. Hereinafter, the term “subject” refers to a human or inferior animal being given Halofuginone. For example, the drug may be administered by the topical (including trans-urethral), oral or parenteral routes.

Formulations for topical administration may include, but are not limited to, lotions, gels, creams, suppositories, drops, liquids, powders or sprays. Conventional pharmaceutical carriers, aqueous, oily or powdery bases, or thickeners and the like may be necessary or desirable.

Compositions for oral administration include powders or granules, aqueous or non-aqueous suspensions or solutions, sachets, capsules or tablets. Thickeners, diluents, flavoring agents, dispersing aids, emulsifiers or binders may be desirable.

Formulations for parenteral administration may include, but are not limited to, sterile aqueous solutions which may also contain buffers, diluents and other suitable additives.

Dosing depends on the severity of symptoms and on the subject's response to Halofuginone. Persons of ordinary skill in the art will readily determine optimal dosages, dosing methodologies and frequencies of dosing.

### EXAMPLE 4

Embodiments for Preventing the Recurrence of Urethral Strictures:

As noted previously, Halofuginone has been shown to be effective in preventing the recurrence of urethral strictures after initial therapy with internal urethrotomy.

The following example is solely illustrative of the embodiments aiming at preventing the recurrence of urethral strictures and is not intended to be limiting. The embodiment includes the step of administering Halofuginone, in combination with a pharmaceutically acceptable carrier such as described in Example 3 above, to a subject to be treated.

Halofuginone is administered to a subject according to an effective dosing methodology immediately after primary treatment of urethral stricture by internal urethrotomy, urethral dilatation or surgical urethroplasty, for a sufficient period of time to optimize efficacy and prevent recurrence of urethral stricture.

### EXAMPLE 5

Manufacturing Process of a Drug Containing Halofuginone:

The following is an example of a process for manufacturing Halofuginone. First, Halofuginone is synthesized according to Good Pharmaceutical Manufacturing Practices (GPMP). Examples of processes for the synthesis of Halofuginone and its related quinazolinone derivatives are given in U.S. Pat. No. 3,338,909. Next, Halofuginone is combined with a suitable pharmaceutical carrier, as described in Example 3 above, again in accordance with GPMP.

Whereas the invention has been described by referring to a limited number of embodiments, it will be appreciated that many variations, modifications and other embodiments of this invention may be made.

What is claimed is:

1. A method for preventing or inhibiting recurrence of a urethral stricture in a subject, the method comprising:
topically administering a gel comprising halofuginone to a urethra of a subject in recognized need of prevention or inhibition of urethral stricture recurrence.

2. The method according to claim 1, wherein the gel is administered to the subject by topical intra-urethral application.

3. The method according to claim 1, wherein the subject was previously treated with conventional therapy for urethral stricture.

4. The method according to claim 1, wherein the method is performed subsequent to internal urethrotomy, urethral dilatation, or surgical urethroplasty.
